(19) United States
(12) Patent Application Publication
(10) Pub. No.: US 2022/0041967 A1
(43) Pub. Date: Feb. 10, 2022

(54) REAL-TIME MONITORING OF SINGLE CELL OR EVENTS

(71) Applicant: Thunderbio Innovation Ltd, Hong Kong (CN)

(72) Inventors: Ruyuan SONG, Huanghua (CN); Xiaonan XU, Jieyang (CN); Shuhuai YAO, Hong Kong (CN); Kwok Fai Joseph CHOW, Hong Kong (CN)

(21) Appl. No.: 17/276,063

(22) PCT Filed: Sep. 18, 2019
(86) PCT No.: PCT/CN2019/106356

§ 371 Date: Mar. 12, 2021

Related U.S. Application Data
(60) Provisional application No. 62/733,790, filed on Sep. 20, 2018.

(51) Int. Cl. C12M 3/06; C12M 1/12; C12M 1/34

(52) U.S. Cl. CPC C12M 23/16; C12M 41/46; C12M 41/12; C12M 25/16

(57) ABSTRACT

The present invention relates to methods and devices for monitoring events occurred in a single cell or examining cell characteristics in a single cell in a massive parallel and real-time manner. In one embodiment, the present invention provides a single-cell culturing system for culturing and monitoring a large number of cells independently at single-cell level. In one embodiment, the present invention provides methods and devices for studying or monitoring single-cell response to an external stimulus in a massive parallel and real-time manner. In one embodiment, the present invention provides methods and devices for studying or monitoring drug response at single-cell level in a massive parallel and real-time manner.

### CROSS REFERENCE TO RELATED APPLICATION

The present application claims priority of U.S. Patent provisional Application No. 62/733,790, filed on Sep. 20, 2018. The content of this application including all tables, diagrams and claims is incorporated hereby as reference in its entity.

### FIELD OF THE INVENTION

The present invention relates to a method and/or device for monitoring events or examining characteristics in a single cell using a microfluidic platform in a real-time manner.

### BACKGROUND OF THE INVENTION

Conventional cells studies investigate physiological traits on averages of cell ensembles in the order of 10³-10⁶ cells, thereby unveiling only the average genotypic/phenotypic traits of the population. However, ty (i.e., a phenomenon of cell-to-cell variation within a population of seemingly identical cells) is in fact a general feature of biological systems and has been observed across all levels of life, from single bacterial cells to human tissues.

More importantly, diseases often originate from abnormalities in a small minority of cells within an organism. The analysis of single cells in large enough numbers can reveal cellular genetic/phenotypic heterogeneity in the genomic alteration, and responsiveness to environmental stimuli and chemotherapeutic stimuli at high resolution, which is important for the understanding of molecular mechanisms underlying cellular function and dysfunction, targeting specific cell type, drug screening, genetic analysis, enzyme analysis and early diagnosis of diseases. Conventional methods of single-cell analysis relying on well-plates and robotics can only handle and analyze a small number of cells due to their high cost and complexity. Ultrahigh-throughput methods adapted to characterize millions of cells are highly demanded, especially when the type of cells that is important for analysis or screening exist in very low abundance in the sample.

Droplet microfluidics techniques which provide monodisperse aqueous micro-compartmentalization for isolating single cells and reagents in a very high-throughput way allows efficient processing and analysis of tens of thousands to millions of cells. Besides, the low volumes of the droplets make very large screens economically available. Emulsion polymerase chain reaction (ePCR) which can perform massive parallel single copy PCR reaction by partitioning nucleic acids (DNA or RNA) into small droplets dispersed in an oil phase provides a powerful tool for high-throughput genetic detection in single cells and hence realizes single-cell analysis. Droplet microfluidics-based single-cell analysis currently play an increasingly significant role in elucidating the heterogeneities of cell populations and their underlying causes.

Laser-based flow cytometry has been used for single-cell analysis on phenotypic traits, such as enzyme, biomarkers, and responsiveness to drug screening. It uses laser light to analyze the presence of fluorescent molecules and light-scattering properties of single cells as they pass a detector in single file fashion at a rate of tens of thousands of cells per second. Fluorescence microscopy is a more dynamic method. Fluorescence microscopy of cells immobilized in microfluidic devices opens up many new possibilities for single-cell studies because the environment that the single cells are subject to could be precisely controlled and modified in the microfluidic devices. However, some important single-cell analyses, such as tracking of specific cells over time, analysis of secreted products and analysis of isolated cells or clones, have been beyond the purview of flow cytometry and fluorescence microscopy methods due to the lack of a robust compartmentalization of single cells by these techniques.

For single-cell analysis on genotypic characterization, individual cells are isolated into each well of the well-plate via a fluorescence-activated cell sorting system. The individual cells undergo DNA/RNA extraction, amplification via polymerase chain reaction (PCR) or reverse transcription polymerase chain reaction (RT-PCR) and genetic detection or whole-genome sequencing to obtain the genetic information of individual cells. This method can only handle and analyze a small number of cells due to their cost and complexity.

Recently, with high throughput and excellent controllability of droplet microfluidics, massive parallel single-cell PCR or RT-PCR can be performed in microfluidic droplets for single-cell genetic analysis. Such method relies on the end-time detection to identify rare mutant genes of each cells, as the digital-PCR technique does. However, genetic information like messenger RNA (mRNA) of each cell across the cell population varies not only in terms of the absence or presence of expression but also in the expression level, while current techniques fail to discriminate mRNA expression level in quantitative way. More importantly, a method capable of determining and analyzing both the phenotypic and genotypic characteristics of single cell in tandem is still lacking at the time of this invention.

This invention introduces for the first time the concept of using a microfluidic platform for monitoring events in a single cell or examining cell characteristics in a single cell in a real-time manner, thereby allowing a more in-depth understanding of molecular mechanisms underlying cellular function and dysfunction.

### SUMMARY OF THE INVENTION

In one embodiment, the present invention provides methods and devices for monitoring events occurred in a single cell or examining cell characteristics in a single cell in a massive parallel and real-time manner.

In one embodiment, the present invention provides a single-cell culturing system for culturing and monitoring a large number of cells independently at single-cell level, comprising partitioning a population of cells into many single microgel molecules, where each of the microgel molecules contains a single cell and is loaded into an incubation chamber for incubation and subsequent assays or analysis.

In one embodiment, the present invention provides methods and devices for studying or monitoring single-cell response to an external stimulus in a massive parallel and real-time manner.

In one embodiment, the present invention provides methods and devices for studying or monitoring drug response at single-cell level in a massive parallel and real-time manner.

### BRIEF DESCRIPTION OF DRAWINGS

FIG. 1 is a schematic diagram showing one embodiment of the present invention for analyzing the level of messenger RNA (mRNA) in single cells. The left panel shows the process of encapsulation of a cell and other reagents. Individable cell in a cell suspension, lysis buffer for cell lysis and reagents and primers for reverse transcription-polymerase chain reaction (RT-PCR mix) are mixed and encapsulated in a single droplet. The right panel shows the process of detection and quantification of single-cell mRNA by real-time monitoring of the reverse transcription polymerase chain reaction (RT-PCR) process. The process includes cell lysis, reverse transcription and PCR reaction which are performed at single cell level in a massive parallel manner, and the monitoring is real-time since target-specific fluorescent signals in each droplet are measured during the PCR.

FIG. 2 shows one embodiment of the present invention for establishing a microgel-based cell culturing system, comprising a chamber for cell incubation, inlets for introducing fluids into the chamber and outlets for removing fluids (e.g. waste) or collecting cells from the chamber.

FIG. 3 is a schematic diagram showing one embodiment of the present invention for establishing a microgel-based cell culturing system which can be used to conduct massive parallel monitoring and analysis at single-cell level. The left panel shows the process of encapsulating of cells and agarose solution. Individual cell in a cell suspension and agarose solution are mixed and encapsulated collectively in a single microgel. The right panel depicts the top view of a U-shaped array for holding individual droplets. Microgels are first loaded and released to the U-shaped array of the incubation chamber, excessive oil is then removed from the microgels through washing, and cell culture is carried out in the incubation chamber under normal culture conditions with medium perfusion. In one embodiment, the present culturing system may take the form as depicted in FIG. 2.

FIG. 4 shows the results of real-time digital PCR of droplets throughout the entire PCR process comprising 45 PCR cycles using one embodiment of the present invention. The fluorescent intensity of droplets was measured in each cycle and 5000 droplets were counted in each measurement.

FIG. 5 shows one embodiment of droplet generation in the present invention.

FIG. 6 shows another embodiment of droplet generation in the present invention.

FIG. 7 shows one embodiment of a droplet generating device comprising a flow focusing structure coupled downstream with a droplet storage chamber.

FIG. 8 shows one embodiment of anchoring structures in a droplet incubation chamber. The anchoring structure strap individual droplets at pre-determined positions in the droplet incubation chamber.

FIG. 9 shows another embodiment of an anchoring structure in a droplet incubation chamber.

FIG. 10 shows a florescence image of droplets obtained by a CCD camera.

FIG. 11 shows one embodiment of digital quantification of RNA from a single exosome.

FIG. 12 shows a process of digital quantification of RNA from a single exosome as part of the present invention.

### DETAILED DESCRIPTION OF THE INVENTION

This invention provides methods and devices for monitoring events occurred in a single cell or examining cell characteristics in a single cell in a massive parallel and real-time manner.

The present invention provides methods and devices for monitoring events occurred in a single cell or a single membrane-bound organelle in a massive parallel and real-time manner.

The present invention provides methods and devices for monitoring phenotypic characteristics and/or genotypic characteristics of a single cell in a massive parallel and real-time manner.

In one embodiment, the present invention provides a microfluidic platform that is capable of generating thousands of droplets, thereby compartmentalizing a cell-containing sample into thousands of isolated droplets, each containing a single cell or a single membrane-bound organelle.

In one embodiment, the present invention provides a droplet incubation chamber for accommodating and incubating droplets containing single cells or single membrane-bound organelles, thereby allowing parallel independent reactions to be carried out in each of the droplets simultaneously and monitoring the process in each of the droplets in a massive parallel and real-time manner.

In one embodiment, the present invention provides a single-cell culturing system for culturing and monitoring a large number of cells independently at single-cell level. In one embodiment, the present single-cell culturing system involves partitioning encapsulating a population of cells into many single microgel molecules, where each of the microgel molecules contains a single cell and is loaded into an incubation chamber for incubation and subsequent assays or analysis.

In one embodiment, the present invention provides methods for culturing a large number of cells independently at single-cell level and studying their characteristics at single-cell level using devices or systems described herein.

In one embodiment, the present invention provides methods for studying or monitoring single-cell response to an external stimulus in a massive parallel and real-time manner. In one embodiment, external stimulus includes environmental stimulus, stress and chemical stimulus.

In one embodiment, the present invention provides methods for studying or monitoring drug response at single-cell level in a massive parallel and real-time manner.

Overall, the present invention is capable of simultaneously conducting independent assays in each cell-containing droplet and monitoring cellular events in each cell or its phenotypic and genotypic characteristics in a real-time and high-throughput manner, therefore is very useful for studying cellular heterogeneity.

### Droplet Generation

In one embodiment, the present invention provides a droplet generating device that is capable of generating thousands of droplets, thereby compartmentalizing a cell-containing sample into thousands of isolated droplets, each containing a single cell or a single membrane-bound organelle.

In one embodiment, the present droplet generating device is a microfluidic platform that is capable of generating partitioning a liquid sample into a high quantity of droplets.

A person having ordinary skill in the art would readily appreciate a variety of droplet generating device of different types and forms is applicable to the present invention, provided that such device is capable of generating droplets suiting the purposes described herein.

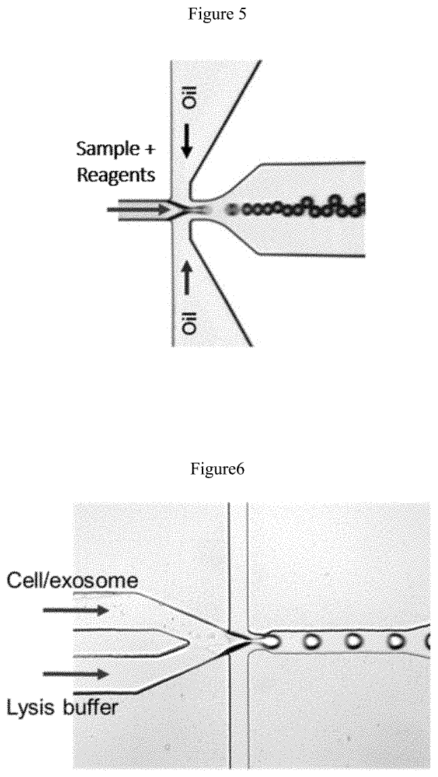

In one embodiment, inlets are provided in the droplet generating device to introduce various liquids (e.g. oil, samples and reagents for carrying out reactions) into the droplet generator. In one embodiment, various liquids for droplet generation are provided to the droplet generator via same inlet. In one embodiment, various liquids for droplet generation are provided to the droplet generator via different inlets. FIG. 5 and FIG. 6 show two embodiments of droplet generation using the present invention. In FIG. 5, the original sample and reagents for carrying out subsequent reactions are premixed and the resulting mixture is subject to the droplet generator for encapsulation. In FIG. 6, since pre-mixing of cells or exosomes and the lysis buffer will lead to lysis of the cells or exosomes, the original sample containing cells or exosomes and lysis buffer are loaded into the droplet generator via different inlets, such that they cannot be brought into contact before they are encapsulated into the droplets.

Droplet generating device of the present invention can be of any structure or system that is capable of partitioning a liquid sample into a large quantity of droplets.

In one embodiment, droplet generating device include but are not limited to structures of flow focusing, crossflowing, co-flowing, step emulsion and micro channel emulsification. P. Zhu and L. Wang (2017) describe a few technologies for droplet generations, the contents of which are hereby incorporated by reference in their entirety into this application.

In one embodiment, the present droplet generator is a shear-based droplet generating device which utilizes shear stress to pinch the fluid thread into small droplets. In one embodiment, shear-based droplet generating devices include but are not limited to devices comprising a cross-flowing structure, a co-flowing structure and a flow focusing structure.

In one embodiment, the present droplet generating device is an interfacial tension-based droplet generating device wherein interfacial tension is the dominant driving force in the process of droplet breakup. In one embodiment, interfacial tension-based droplet generating devices include but are not limited to devices comprising a structure of T-junction combining with step emulsion and a micro-channel emulsification structure.

In one embodiment, the present droplet generating device comprises a droplet generating structure described in WO2016189383A1, the contents of which are hereby incorporated by reference in their entirety into this application.

In one embodiment, methods that are capable of generating droplets can be utilized in the present invention for droplet generation, including but are not limited to high-shear stirring, ultrasonic emulsification, high-pressure homogenization and membrane emulsification.

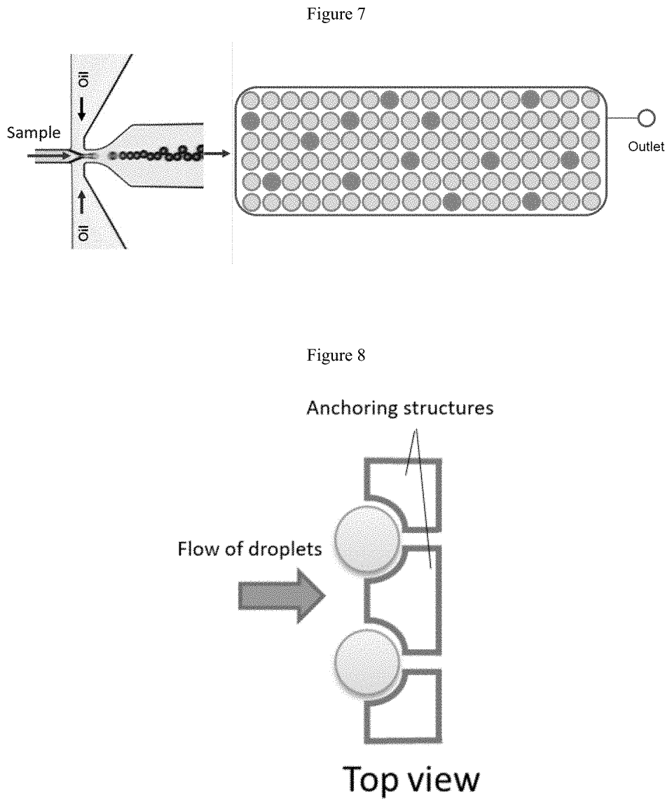

In one embodiment, the present droplet generating device comprises a flow focusing structure which constricts the flow to strength the focusing effect. In one embodiment, the flow focusing structure is a 2D planar flow focusing structure. FIG. 7 shows one embodiment of a droplet generating device comprising a flow focusing structure and a droplet storage chamber for holding the droplets generated. In FIG. 7, the sample at the center channel is shared by fluid from side channels and breaks up into small droplets which are then sucked into the droplet storage chamber due to capillary force.

In one embodiment, the present droplet generating device comprises a crossflowing structure which permits the continuous phase and dispersed phase to intersect at a certain angle θ. In one embodiment, the present droplet generator comprises a structure of T-junction, Y-junction, double T-junction, K-junction or V-junction.

In one embodiment, the present droplet generating device comprises a co-flowing structure in which the dispersed fluid thread is punched off by the surrounding flow continuous phase. In one embodiment, the co-flowing structure is a 2D planar co-flowing structure.

In one embodiment, the present a droplet generating device comprises a step emulsion structure. In one embodiment, the present droplet generating device comprises a step emulsion structure combined with a T-junction structure which is horizontal or vertical

In one embodiment, the present droplet generating device comprises a microchannel emulsification structure.

In one embodiment, components or parts of the droplet generating device which is responsible for droplet generation (i.e. sample compartmentalization) have a hydrophobic surface. It can be accomplished by chemical surface coating by conjugating hydrophobic groups on the surface of the components or parts. In one embodiment, a surfactant such as Span 80, Tween 20 or Abil EM90, perfluoropolyether-polyethylenoxide-perfluoropolyether triblock copolymer (PFPE-PEG-PFPE) is added to the oil phase or water phase to avoid droplet coalescence or prevent molecules such as enzymes, DNA or RNA from adhering to the solid surface or water-oil interface.

In one embodiment, droplets are generated as emulsion droplets and are not limited to a particular type of emulsion. In one embodiment, emulsions include but are not limited to oil-in-water, water-in-oil and water-oil-water double emulsion.

In one embodiment, oil and surfactant are used for droplet generation. In one embodiment, the ratio of surfactant to oil is 1-5% (by weight). In one embodiment, oil to be used for droplet generations includes but is not limited to mineral oil, silicon oil, fluorinated oil, hexadecane and vegetable oil. In one embodiment, surfactant to be used includes but is not limited to Span 80, Tween 20/80, ABIL EM 90 and phospholipids, PFPE-PEG-PFPE. Surfactants that can be used in droplet-based microfluidics have been described by Baret, Jean-Christophe (2012), the content of which is hereby incorporated by reference in its entirety into this application.

In one embodiment, the present droplet generating device is capable of compartmentalize cells into water-in-oil droplets (10-200 μm in diameter) at a frequency of about 0.1 kHz to about 20 kHz. In one embodiment, the frequency for droplet generations is about 0.01 to 1 kHz.

In one embodiment, the present droplet generating device is capable of partitioning millions of cells into individual droplets in minutes. In one embodiment, the present droplet generating device is capable of partitioning millions of cells into individual droplets in about ten minutes.

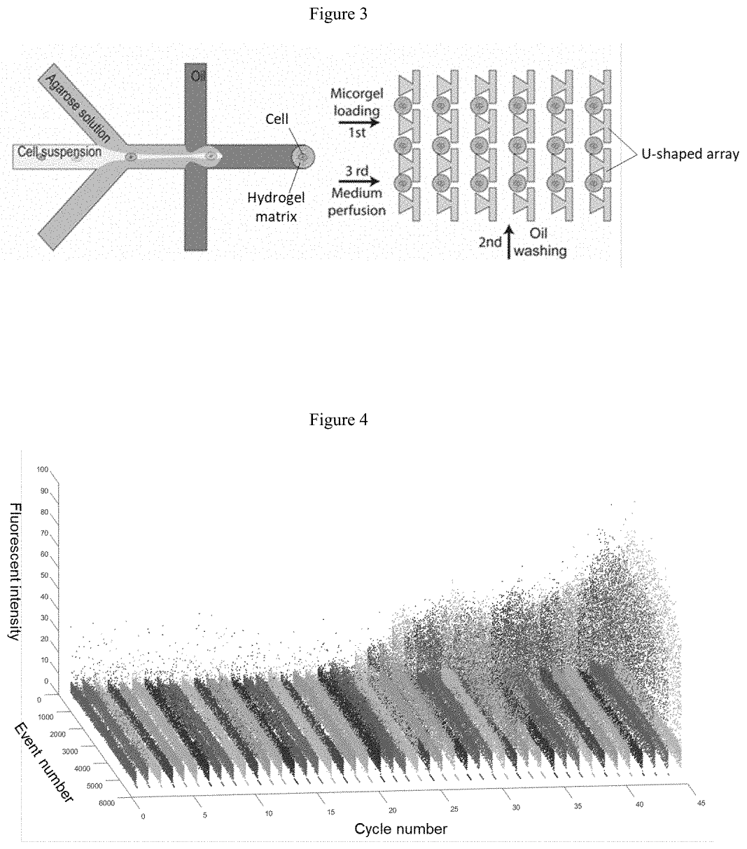

In some embodiments, the present invention provides a generating device capable of generating microgel particles, the device is a microfluidic droplet generator with the first inlet for importing the cell solution, the second inlet for importing gel solution and the third inlet for the oil as the continuous phase. The aqueous phase of the cell solution and the gel solution meets firstly as to form a mixture liquid drop at the junction and then flowing downstream to meet the oil phase as to form a droplet packaged by oil phase. Emulsified into droplets and a microgel particles are formed with temperature decrease within the droplets afterwards. Each of at least a portion of the microgel particles include a single cell. As shown in FIG. 3, the cell is immobilized in the gel matrix of microgel particles.

During the steps of forming mixture liquid droplets and the steps of form the oil phase packaging the liquid droplets therein. These will depend on when the gel solution become solid phase from liquid phase. Normally, in micro-fluid channel, the distance where forming the mixture liquid phase (cell solution mixing with gel solution) is very short to the place where the mixture liquid is packaged by the oil phase downstream of the cell solution. So, when gel solution meets or mixes with cell solution, gel solution at almost does not change from the liquid phase into soil phase in such a short time, and still are the liquid phase. Once or after the mixture liquid droplets are packaged by the oil phase, the gel compounds in the mixture liquid droplets will be changed from liquid phase into solid phase (form micro-gel particles, like matrix with nanopares and the cell are packaged in the matrix) depending on the temperature change, such as from a higher temperature into a lower temperature.

In some embodiments, the device comprises of more inlets for importing different components of gel of some kind, like catalyst, monomer, cross-linker, etc.

In some embodiments, the device further includes an outlet for exporting microgel particles. In some embodiments, the outlet leads to a storage system, and microgel particles are exported from the outlet directly to a storage system for storage or culture. The storage system and cell culture system herein can be interchanged in concept. The incubation chamber has for example, anchoring structures as shown in FIG. 3, and each anchoring structure traps an microgel particle.

In some embodiments, the microgel particle generating device further includes a heating unit, and the heating unit allows the gel solution to maintain a liquid state.

The microfluidic device herein includes a variety of micro fluidic channels and these channels communicate mutually. The gel-packaged or encapsulated cell microparticles o can be fabricated by any structure in the prior art. For example, a crossflowing structure which permits the continuous phase and dispersed phase to intersect at a certain angle θ. In one embodiment, the present droplet generator comprises a structure of T-junction, Y-junction, double T-junction, K-junction or V-junction.

For the microgel particle in the storage chamber or the incubation chamber, the oil phase or the surfactants in the oil phase can be removed in some ways to release the microgel particles into aqueous culture medium. For example, the method described in the Example 4 can be carried out. After removing the oil phase or surfactant in this way, the cell culture medium can be infused through the inlet of the cell solution, the inlet of the gel solution, or the inlet of the oil phase, and the culture medium can diffuse into/out of the microgel particle through the nanopores on the gel matrix to supply nutrients to cells and remove the waste of cellular metabolism.

Of course, some testing substances can be imported from these inlets, for example, the drugs. These drugs are imported into the incubation chamber, and enter the microgel particle through the nanopores on the gel matrix to interact with an individual cell to investigate the cell viability or some specific reactions and achieve real-time testing and monitoring of cellular activity interacted with drugs.

### Droplet Characteristics

In one embodiment, the quantity, size (i.e., diameter), volume and type of emulsion of droplets generated or used by the present invention depend on the subsequent processing or analysis required.

In one embodiment, the number of droplets generated ranges from several hundreds to several millions.

In one embodiment, the size of the droplets generated ranges from about 5 μm to about 200 μm. In one embodiment where cells are compartmentalized, the size of the droplets generated ranges from about 10 μm to about 200 μm.

In one embodiment, the volume of the droplets generated ranges from about 0.65 fL (femtoliter) to about 4 nL (nanoliter).

In one embodiment, droplets generated are of uniform diameter. In one embodiment, droplets generated have a uniform diameter with coefficient of variation less than 5%. In another embodiment, droplets of varying diameters are generated by adjusting the loading pressure.

In one embodiment, each droplet produced by this invention contains no more than one copy of the target molecule (e.g. cell, exosome, or a certain type of biomolecule) to be analysed in subsequent steps. In one embodiment, the number of droplets to be produced and the volume of sample introduced for droplet generations are adjusted in a manner such that each produced droplet would contain no more than one target molecule. Digital methods which distribute target molecules into a large number of droplets theoretically follow the principle of Poisson distribution (Majumdar, 2015). Quantification of target molecules can then be done by counting the droplets which contain one or more copies of the target molecule. To achieve an absolute quantification, each droplet should contain no more than one copy of the target molecule. Generally, according to the principle of Poisson distribution, over 99% of droplets will contain no more than one copy of the target molecule if the ratio of the number of droplets to the number of target molecule is larger than 10, while the percentage will be 96% if the ratio is about 3. For example, when using the present invention for digital quantification of exosome, 10 times more droplets than the expected number of cells is used to ensure that each droplet will capture no more than one target cell for an absolute quantification. Alternatively, in case a single copy of target molecule per droplet is not guaranteed (i.e., some of the droplets may contain more than one copy of the target molecule), Possion statistics are employed to calculate the absolute number of the target molecule (Majumdar, 2015).

In one embodiment, the present droplet generating device is capable of achieving a high dynamic range by generating droplets of size and quantity that are sufficient for an accurate quantification of the target molecules in the sample. Generally, for digital analytical techniques which employ partitions (e.g. droplets) for detecting target molecules, the dynamic range of detection (i.e., the range of the number of target molecule that can be detected accurately using digital analytical technique) is determined by two main parameters: the size and total number of droplets, which are limited by the partitioning capability of the droplet generating device. For example, it was reported that the dynamic range of typical digital PCR is 0-10⁶, meaning that typical dPCR is unable to determine the absolute count of a target nucleic acid molecule in the sample if the level of that target nucleic acid molecule exceeds the limit of 10⁶ copies/μL. From statistics, having 3-10 times more droplets than target molecules will have a higher accuracy in detection but a smaller dynamic range. On the other hand, a larger dynamic range can be achieved by utilizing the Poisson distribution (Majumdar, 2015).

In one embodiment, cell concentration in a sample is adjusted to a level such that over 90% of droplets contain no more than one cell. In one embodiment, the optimal range of cell concentration mainly depends on the type of cells in question and the dimension of the droplet generating device. In one embodiment, cell concentration is adjusted in the range of 50,000-100,000 cells/ml.

### Cell-Containing Samples

The present invention can be applied to any type of samples containing cells from any type of organisms, including but not limited to human, animal, plant, fungi, microorganism such as bacterium and virus.

In one embodiment, cells subject to the present invention are obtained from a biological fluid, tissue, organ or any cell-containing materials originated from an organism.

In one embodiment, sample is a liquid sample obtained directly from a viable organism. In another embodiment, sample is a liquid sample obtained directly from a non-viable organism.

In one embodiment, cells subject to the present invention are obtained from a biological sample including but not limited to blood, plasma, serum, tissues, urine, saliva, fecal matters, smear preparations, and discharges such as tears, sputum, nasopharyngeal mucus, vaginal discharge and penile discharge.

In one embodiment, cells described herein can be of any type, form, stage of development or stage of differentiation. In one embodiment, cells described herein comprise identical or different populations of cells. In one embodiment, cells include somatic cells and germ cells. In one embodiment, cells are fully differentiated cells, partially differentiated cells or undifferentiated cells. In one embodiment, cells are immune cells, stem cells or cancer cells of any kind. In one embodiment, cells are cell cultures of any kind, including suspended cells and adherent cells from any types of organisms.

In one embodiment, in additional to cells, the present invention is also applicable to cell-like molecules including but not limited to membrane-bound organelles or cell-derived vesicles such as exosomes.

### Droplet Incubation Chamber

In one embodiment, the present invention provides a microfluidic system comprising a droplet incubation chamber for incubating the droplets, one or more inlets for introducing fluids into the droplet incubation chamber and one or more outlets for removing fluids or cells from the droplet incubation chamber. In one embodiment, the present microfluidic system takes the form of FIG. 2.

In one embodiment, the present invention provides a droplet incubation chamber for accommodating and incubating droplets containing single cells or single membrane-bound organelles, thereby allowing parallel independent reactions to be carried out in each of the droplets simultaneously and monitoring the process in each of the droplets in a massive and real-time manner.

In one embodiment, after the step of droplet generation, droplets generated are loaded to a droplet incubation chamber for further processing and observation.

In one embodiment, droplet incubation chamber described herein is any module that is capable of accommodating droplets, including but not limited to droplets that are generated by the droplet generators.

In one embodiment, droplet incubation chamber described herein is any module that is capable of accommodating droplets, and further allowing parallel reactions or assays to be carried out in the droplets in a controlled manner.

In one embodiment, the design of the present droplet incubation chamber depends on the total number of droplets, volume of droplets, type of cells encapsulated in the droplets and type of reactions or assays to be performed in the subsequent steps.

In one embodiment, the present droplet incubation chamber is a microfluidic chip onto which a high number of droplets can be loaded and incubated therein.

In one embodiment, the present droplet incubation chamber is coupled with the present droplet generating device in a way that droplets generated are sucked into the droplet incubation chamber by capillary force. In one embodiment, droplets are dispersed in the droplet storage chamber such that the droplets are packed in a specified manner. In one embodiment, droplets are dispersed in the droplet storage chamber such that the droplets are loosely or randomly packed.

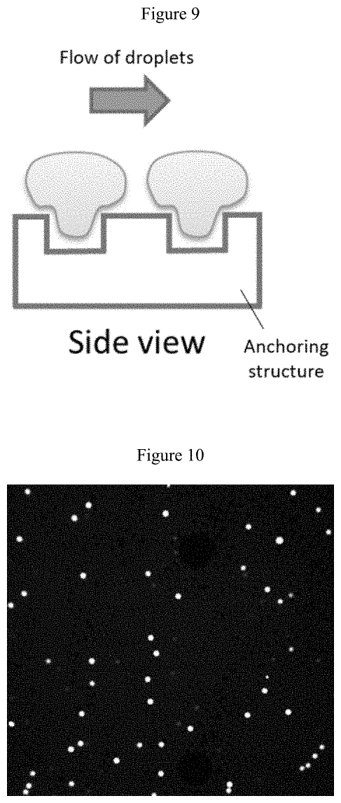

In one embodiment where droplets are dispersed in a specific or pre-determined manner, the droplet storage chamber comprises rows of anchoring structure for anchoring the droplets to pre-determined positions in the droplet incubation chamber. In one embodiment, the anchoring structure takes the form of pillars such as posts arranged in a way that is capable of trapping individual droplets (FIG. 8). As the droplets travel through the droplet incubation chamber, they will be trapped in space between the pillars. In one embodiment, the anchoring structure takes the form of grooves which trap individual droplets by interfacial tension (FIG. 9). In one embodiment, the present droplet incubation chamber comprises anchoring structures or equivalents described in the art, such as those described in Abbyad (2010) and Huebner (2008), the contents of which are hereby incorporated by reference in their entireties into this application.

In one embodiment wherein droplets are randomly packed, no anchoring structures are provided in the droplet storage chamber.

In one embodiment, the present droplet incubation chamber comprises a temperature-controlling unit for regulating the temperature of the droplet incubation chamber. In one embodiment, the temperature is controlled at a temperature that is required for performing a particular assay within the droplets. In one embodiment where the droplet incubation chamber is used for cell culturing, the temperature is controlled at a temperature that is required for culturing the cells within the droplets (e.g. 37° C.).

In one embodiment, the present droplet incubation chamber comprises a gas-controlling unit for maintaining the level of oxygen (O₂) and carbon dioxide (CO₂) in the droplet incubation chamber. In one embodiment, the levels of oxygen (O₂) and carbon dioxide (CO₂) are maintained at a level of 20% and 5% respectively.

In one embodiment, the dimension of the droplet incubation chamber is selected in order to hold the actual or expected quantity of droplets and is compatible with subsequent assays or cell culture to be conducted therein. In one embodiment, the height of the droplet incubation is about 70 μm to 300 μm. In general, single cell analysis requires a lower droplet incubation chamber while culture of spheroids requires a higher droplet incubation chamber.

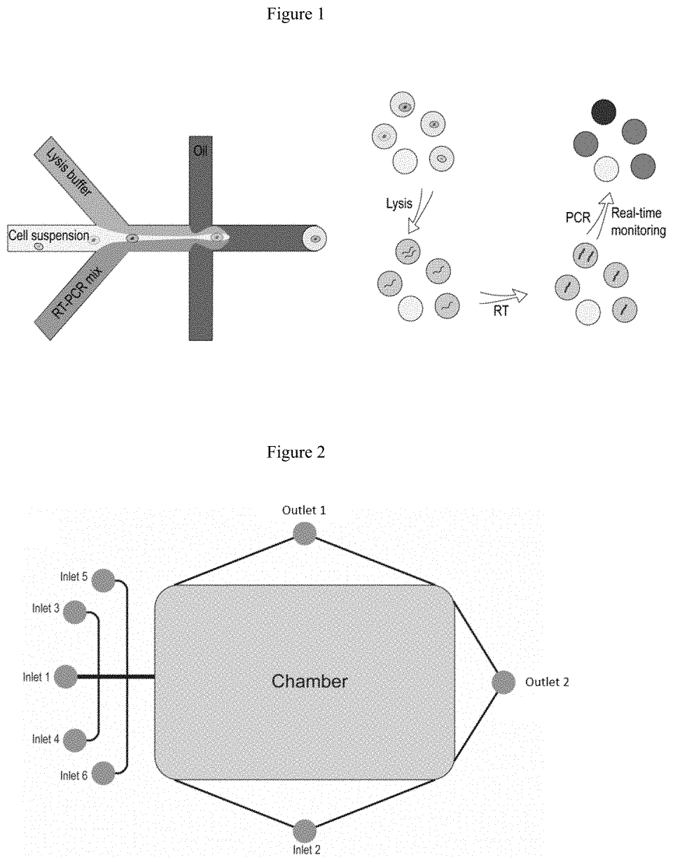

In one embodiment, where the sample and reagents such as buffers, primers, probes and enzymes for performing reactions or assays do not have chemical reactions, they can be premixed and loaded are encapsulated in droplets together with the cells such that independent parallel reactions can be carried out in the droplets immediately after being loaded into the droplet incubation chamber. In one embodiment where the sample and reagents do not have chemical reactions, they can be premixed and loaded into the droplet generating device as a mixture through one inlet. In another embodiment where the sample and one or more of the reagents react, these reagents and sample cannot be introduced to the droplet generating device as a mixture but loaded into the droplet generator through different inlets and compartmentalized into droplets at the junction of the droplet generating device. As illustrated in FIG. 1 and Example 2, lysis buffer, RT-PCR mix (including primer, TaqMan probes or other reagents for RT-PCR) and cell suspension are provided to the droplet generators separately to prevent pre-mature cell lysis. These reagents are loaded into the droplets along with cells at the time of encapsulation and will be used for mRNA detection and quantification of single cell via RT-PCR. The exact reagents to be used and their concentrations and volumes will depend on the requirements of reactions or assays to be performed.

### Microfluidic Channels

In one embodiment, the present device comprises a plurality of microfluidic channels for delivering fluids to and from various components of the device. In one embodiment, the present droplet generating device, droplet incubation, outlet and/or other components described herein comprises one or more microfluidic channels which set the flow paths of the fluids within these components. In one embodiment, one or more microfluidic channels are provided between different components (e.g. between droplet generating device and droplet incubation chamber) so as to direct fluid from one component to another component. In one embodiment, the exact type or configuration (e.g. structure, length, diameter, number of branches and density) of the microfluidic channels to be used depends on the purpose of having the microfluidic channels and the desirable flow resistance of individual components.

In one embodiment, microfluidic channels are made of materials selected from the group consisting of silicon, glass, plastics and polydimethylsiloxane (PDMS).

In one embodiment, the same type or configuration of microfluidic channels is used in various components described herein. In another embodiment, various types or configurations of microfluidic channels are used in various components described herein.

In one embodiment, the present droplet generating device comprises two microfluidic channels for delivering oil and one or more microfluidic channels for delivering sample fluid and/or reagents. In one embodiment, the actual configuration depends on the type of emulsion chosen and the number of inlets required.

In one embodiment, a microfluidic channel is used to connect the droplet generating device with the droplet incubation chamber. In one embodiment, the microfluidic channel has a diameter 1-2 times the diameter of a droplet. Generally, a larger diameter of the microfluidic channel helps to stabilize the droplets as they pass through the channels, and constricting the fluid flow within the channel will also help to stabilize the droplets.

In one embodiment, the droplet incubation chamber does not have any microfluidic channel and droplets generated will self-assemble to spread on the flat surface of the chamber. In cases where wells are present in the droplet incubation chamber, droplets will be spread in the chamber and then guided into the wells by interfacial tension.

In one embodiment, the present outlet comprises a microfluidic channel which has a diameter of up to several hundred micrometers.

In one embodiment, the microfluidic channels are rectangular in shape (i.e., have a rectangular cross-section). In another embodiment, the microfluidic channels have a round cross-section.

### Multiplex Reactions in Multiple Droplets and Detection System

As illustrated herein, the present invention provides a platform for carrying out multiplex reactions in all droplets containing single cells and conduct measurements in a real-time manner. Different from the end-point measurement in existing droplet-based technologies, this approach can provide a real-time monitoring and analysis of cellular events and cellular characteristics in question.

In one embodiment, the present invention provides device and method for carrying out multiplex reactions or assays in droplets containing single cells. By carrying appropriate reactions or assays, events occur in each single cell and phenotypic and/or genotypic characteristics of each single cell can be monitored and analyzed according to the description described herein.

In one embodiment, after partitioning a sample (and reagents if any) into numerous isolated droplets and loading the droplets into the droplet incubation chamber, the present device and method carry out one reaction per single droplet for every droplet in the droplet incubation chamber concurrently. The present invention permits reactions in one droplet to be carried out independent of any other reactions in other droplets, therefore allowing independent monitoring and analysis of events occur in cells or characteristics of cells at single-cell level.

In one embodiment, reactions are wholly or part of any compatible bioassay used in the art. In one embodiment, reactions to be carried out are chosen depending on the nature of the target bio-molecules.

In one embodiment, reagents for carrying out the reactions are mixed with cell-containing sample at the time of droplet generation, thereby producing droplets containing both the cells and reagents. In another embodiment, reagents for carrying out the reactions are introduced to the cell-containing droplets after they are generated and loaded into the droplet incubation chamber.

In one embodiment, reactions to be carried out in the droplets within the droplet incubation chamber are reactions that introduce signals specific to or otherwise indicative of events or cell characteristics to be monitored. In one embodiment, signal-generating moieties that generate detectable signals specific to or otherwise indicative of the events or cell characteristics to be monitored are included in the reactions.

In one embodiment, signal-generating moieties are specific to a bio-molecule. In one embodiment, signal-generating moieties include but are not limited to chemiluminescent, fluorescent, chromomeric substrates, or other substrates that is convertible to a product capable of being detected.

In one embodiment, type of signal-generating moieties and their amounts to be used depend on the events or cell characteristics to be monitored, and biomolecules to be detected or quantified if applicable.

In one embodiment, target-specific compositions are included in the reactions so as to recognize and label target biomolecules in the droplets. In one embodiment, target-specific compositions are molecules that can specifically recognize a target biomolecule by means of structural recognition, functional recognition, or both.

In one embodiment, target-specific compositions are used to identify and label a specific type or species of biomolecule in the droplets.

In one embodiment, the biomolecule is a nucleic acid, a protein or a small molecule.

In one embodiment, the biomolecule is a cell-free molecule including but is not limited to a cell-free DNA (cfDNA), a cell-free protein, an exosome and a cell-free molecule circulating in the body fluid of the subject. In one embodiment, the biomolecule is a molecule attached to the surface of a cell or included in a cell.

In one embodiment, the biomolecule is a nucleic acid of various types (e.g. DNA including cDNA, RNA including mRNA and rRNA), forms (e.g. single-stranded, double-stranded, coiled, as a plasmid, non-coding or coding) and lengths (e.g. an oligonucleotide, a gene, a chromosome and genomic DNA).

In one embodiment, the biomolecule is a protein which is a peptide or a polypeptide, including an intact protein molecule, a degraded protein molecule and digested fragments of a protein molecule. In one embodiment, biomolecules include but are not limited to antigens, receptors and antibodies.

In one embodiment, the biomolecule is a small molecule such as a metabolite. In one embodiment, the metabolite is a disease-related metabolite which is indicative of the presence or extent of a disease or a health condition. In one embodiment, the metabolite is a drug-related metabolite such as a drug by-product of which the level changes in a subject body consuming the drug.

In one embodiment, the biomolecule is a molecule produced by a tumor or cancer, or by the body of the subject in response to a tumor or cancer.

In one embodiment, the biomolecule is not normally found in healthy subject. In one embodiment, the biomarker is a molecule that is normally found in a healthy subject but the level of which is indicative of a particular disease or a health condition.

In one embodiment, target-specific compositions are primers or probes comprising nucleic acids that contain sequence complementary to the target nucleic acids. In one embodiment, target-specific compositions are probes, antibodies or equivalents that recognize specific epitopes or spatial configurations possessed by a target biomolecule such as protein, peptide and viral particle.

In one embodiment, target-specific compositions are molecules that can be processed (e.g. digested, reduced, oxidized, or otherwise modified) by the target biomolecules. For example, where an enzyme is the target biomolecule, target-specific compositions can be a small-molecule substrate that is subject to the enzymatic reaction catalyzed by that enzyme.

For example, biomolecules that are nucleic acids may require amplification by polymerase chain reaction (PCR) and labelling by complementary probes, while biomolecules that are proteins may require hybridization using antibody that recognizes certain epitopes of the proteins.

In one embodiment, where nucleic acids are to be detected and quantified, reactions include but are not limited to polymerase chain reaction (PCR), reverse transcription-PCR (RT-PCR), real-time PCR, and real-time RT-PCR, reverse transcription, labeling, digestion, blotting procedures, enzyme-linked immunosorbent assay (ELISA), radioimmunoassay (RIA), immunoassays and enzymatic assays.

For example, ddPCR™ EGFR Exon 19 Deletions Screening Kit (Bio-Rad Laboratories, Inc.) is used to screen for mutations of 15 deletions in Exon 19 of the EGFR gene. Other deletions in this region of the EGFR Exon 19 may also be detected by this kit. EGFR Exon 19 deletions are commonly associated with melanoma, colorectal, and lung cancers. Examples 2 and 3 describe detection and quantification of RNA molecules using the present invention.

In one embodiment, where biomolecules of protein nature (e.g. protein, peptide, antibody) are to be detected and quantified, reactions include but are not limited to ELISA-based reactions, labeling of target protein by target-specific signaling moiety and reactions that are catalyzed or inhibited by the target protein.

In one embodiment, antibody conjugated with specific customized DNA strands, immunostaining and real-time PCR with TaqMan™ probes are used for protein detection. The proteins on the cell membranes are firstly labeled by an antibody which recognize the target proteins and conjugated with specific DNA strands via antibody-antigen interaction. The cells are compartmentalized individually into droplets supplemented with Platinum Multiplex PCR Master Mix (Thermo Fisher, USA), TaqMan™ probes which recognize the DNA strands, and droplet stabilizers for real-time PCR detection. The DNA strands are then amplified via PCR and the DNA strands are detected by real-time PCR with TaqMan™ probes.

In one embodiment, reactions for detecting and quantifying exosomes include but are not limited to reactions for labeling, detecting or quantifying exosome-specific biomolecules. In one embodiment, absolute count of exosomes can be determined digitally using ExoELISA method. In one embodiment, the method used is described in Liu (2018), the content of which is hereby incorporated by reference in its entirety into this application.

In one embodiment, reactions for detecting and quantifying bacteria include but are not limited to reactions for labeling, detecting or quantifying biomolecules such as DNA, RNA or antigen that are specific to the bacteria in question.

### Detection System and Digital Detection and Quantification of Target Biomolecules

In one embodiment, the present droplet incubation chamber is coupled with a detection system or devices (e.g. an optic system) for collecting signals that are indicative of a cellular event, a cell characteristic or otherwise, thereby allowing a real-time monitoring of the events or examining cell characteristics in tens of thousands of single cells in a parallel and real-time manner.

In one embodiment, the present method comprises a step of measuring the absolute count of signals indicating the presence of target biomolecules and thereby quantifying the target biomolecules in an absolute count.

In one embodiment, the present method comprises a step of quantitatively and independently measuring a specific signal from a plurality of droplets. In one embodiment, the measurement is digital. Digital means the signal is either one or zero. For instance, the droplets with fluorescence are named as ‘positive’ (i.e., the droplets contain target molecule) and the droplets without fluorescence are ‘negative’ (i.e., no target molecule is present in the droplets).

In one embodiment, the present detection system is any system that is capable of capturing, detecting, measuring and/or quantifying signals observed from each droplet in the droplet incubation chamber, including but not limited to signals generated by signal-generating moieties described herein.

In one embodiment, signals are captured, detected, measured and/or quantified continuously during the entire monitoring process. In one embodiment, signals are captured, detected, measured and/or quantified regularly at specified time intervals. In one embodiment, time interval is by second, by minute, by hour or by day. Typically, the scanning rate (i.e., rate of signal detection) for monitoring a cell culture is lower (e.g. every day) than the scanning rate for detection or quantification of biomolecules in single cells (e.g. every two minutes for detection of mRNA molecules).

In one embodiment, signals to be detected are fluorescent signals and systems or devices that are capable of capturing fluorescent signals and measuring the intensity of fluorescent signals are used. In one embodiment, a charge-couple device (CCD) is used to capture fluorescent signals and generates images of florescent droplets deposited in a chamber or on a chip. By counting the number of fluorescent droplets and intensity of fluorescent signals in each of the droplets, the florescent signals can be processed and analyzed. FIG. 10 shows a florescence image of droplets obtained by a CCD camera. In one embodiment, florescent signals measured are processed and analyzed using a proprietary image processing code. In one embodiment, the present proprietary image processing code is capable of processing and decoding florescent signals simultaneously detected from a large number of targets (e.g. 3,000 to 10,000 targets) and outputting florescent signals in each cell.

In one embodiment, an optic system is provided for detecting a plurality of fluorescent signals. In one embodiment, the optic system comprises a device that can measure or collect fluorescent signals including but is not limited to a CCD. In one embodiment, the optic system comprises multiple laser or light-emitting diode (LED) sources for inducing fluorescence or providing visible lights and multiple filters for separating waves or particles of different wavelengths, thereby selectively detecting signals of a particular kind of wave or particle. In one embodiment, the optic system permits change of filter via automation, hence making detection more efficient.

In one embodiment, only one type of biomolecule is detected and quantified per single droplet. In one embodiment, two or more types of biomolecules are detected and quantified per single droplet. For example, protein, nucleic acids, exosomes and/or other type of biomolecules are detected and quantified one after another in one single droplet.

In one embodiment, two or more species of biomolecules of the same type are detected and quantified per single droplet. For example, two or more species of nucleic acids (e.g. a DNA molecule and a RNA molecule) are detected and quantified per single droplet.

In one embodiment, when two or more types of biomolecules are to be detected and quantified per single droplet, one type of biomolecules is first detected and quantified per single droplet, then another type of biomolecules is detected and quantified per single droplet, and so forth. For example, one or more species of nucleic acids are first detected and quantified per single droplet, and then one or more species of peptides are detected and quantified per single droplet thereafter.

In one embodiment, the type of biomolecules detected and quantified in one droplet is different from the type of biomolecules detected and quantified in another droplet.

In one embodiment, the present invention detects 1-5 types of biomolecules per run. In another embodiment, the present invention detects 6-10 types of biomolecules per run. In yet another embodiment, the present invention detects 11-20 types of biomolecules per run.

In one embodiment, the present invention detects 1, 2, 3, 4, 5, 6, 7, 8, 9 or 10 types of biomolecules per run.

In one embodiment, the present droplet generating device, droplet incubation chamber and detection system described herein are provided and function as an integrated unit under full automation, thereby allowing compartmentation of cells, incubation of droplets of cells, performance of reactions in these droplets of cells and detection of signals to be carried out closely one after another.

### Single-Cell Culturing System

In one embodiment, the present invention provides a single-cell culturing system for culturing and monitoring a large number of cells independently at single-cell level.

In one embodiment, the present single-cell culturing system is implemented by partitioning and encapsulating a population of cells into many single microgel molecules. The microgel molecules are then loaded into a cell incubation chamber for incubation and subsequent assays or analysis. In one embodiment, each microgel molecule contains a single cell. In another embodiment, each microgel molecule contain no more than a single cell.

In one embodiment, over 90% of microgel molecules contain a single cell. In another embodiment, over 95% of microgel molecules contain a single cell.

FIG. 3 is a schematic showing one embodiment of the present invention for establishing a microgel-based cell culturing system which can be used to perform massive parallel monitoring and analysis at single-molecule level. The left panel shows the process of encapsulation of cells and gel solution. The individual cell in the cell suspension and gel solution are mixed and encapsulated collectively in a single microgel particle using a water-in-oil emulsion.

At first, the gel solution is prepared by dissolving the agarose powder in solution at an elevated temperature. The temperature of the resulting gel solution is then adjusted or the flow ratio of the gel solution to the cell solution is adjusted so that the gel solution remains liquid while its temperature is not too high to damage the cells. After the gel solution is mixed with the cell suspension, the temperature of the mixed solution drops immediately and gelation occurs, thereby trapping the cells in the gel matrix and forming microgel particles.

The resultant microgel particle is suspended in the oil phase as shown in FIG. 3. At this time, there is an oil phase package between individual microgel particles so that the packed cells will not interact with each other during flow and distribution to avoid cross reaction. In addition, since the microgel particle per se allow diffusion of water, some nutrients can be absorbed into the microgel particle through the gel matrix to supply the nutrients necessary for the growth of cells.

When microgel particles are distributed into a single cell incubation chamber, for example, there are thousands of cell trapping structures in a microfluidic chip, and each trapping structure stores a microgel particle, as shown in the right side of FIG. 3, then the oil phase or residual liquid outside of the microgel particle is removed by washing and only the microgel particle is left in the incubation chamber. By this way, nutrients can be continuously imported from the inlets and these nutrients enter into the gel matrix trapping the cells for the growth of cells through the nanopores on the gel (some nanopores formed by the gel itself), thus, cells can be tested in a dynamic and real-time manner during the cell growth process. These tests include testing on activity, drug response, and internal life activity. These tests can be carried out on thousands of single cells to obtain multiple test results at one time. Each test corresponds to an independent cell.

The right panel shows a number of processes including loading the microgels which are in the form of water-in-oil emulsion to the incubation chamber, oil washing and medium perfusion. Microgels are loaded and released to the U-shaped array in the incubation chamber, oil is removed from the microgels through washing to permit aqueous solutions to enter and leave the microgels and cell culture is carried out in the incubation chamber under normal culture conditions with medium perfusion. In one embodiment, the U-shaped array is an anchoring structure which may take the form of grooves which trap individual droplets by interfacial tension as shown in FIG. 9.

In one embodiment, the present microgel-based cell culturing system comprises the system as illustrated in FIG. 2.

In one embodiment, the present microgel-based cell culturing system comprises droplet incubation chamber described herein.

In one embodiment, cell-containing microgels, media for cell culture or other reagents required for cell culture or assays are introduced into the cell incubation chamber through the inlets. In one embodiment, fluids such as used culture media containing wastes from the cells, microgels or cells are removed from the cell incubation chamber through the outlets.

Example 4 shows one example of using the present invention to compartmentalize individual cells in agarose microgels for long-term incubation.

In one embodiment, hydrogel materials form a hydrogel matrix which blocks the immigration of cells while allowing small molecules (e.g. nutrients, metabolic wastes) to diffuse freely in and out of the cells.

In one embodiment, hydrogel materials that are capable of forming a hydrogel matrix to encapsulate individual cell molecules can be used. In one embodiment, the hydrogel material is agarose. In one embodiment, the hydrogel material is alginate which will undergo gelation upon addition of calcium ion to the alginate solution.

In one embodiment, the pore size of the hydrogel matrix is much smaller than the dimension of cells to be encapsulated therein yet large enough for nutrients and waste to pass through. In one embodiment, the pore size of the hydrogel matrix is about 100× smaller than the dimension of cells. For example, the pore size of the hydrogel matrix is about 100 nm while the dimension of cells is 10 μm. Pore size of the hydrogel matrix can be adjusted by the concentration of the hydrogel solution. A higher concentration of hydrogel will result in a smaller pore size of the resulting hydrogel matrix.

In one embodiment, cell culture conditions and medium used for cell culture using the present invention are similar to those used in a normal cell culture using conventional cell incubation. In one embodiment, temperature and level of oxygen and carbon dioxide are regulated at a level that are suitable for culturing the cells in question.

In one embodiment, the inlets and outlets connected to the cell incubation chamber are driven by one or more pumps (e.g. peristaltic pumps) in order to drive the fluids or microgels into and out of the cell incubation chamber.

In one embodiment, fresh medium is pumped into the cell incubation chamber for nurturing the encapsulated cells in the cell incubation chamber. In one embodiment, medium containing wastes and unused nutrients are removed from the cell incubation chamber through the outlet driven by the pump so as to prevent toxic substances from accumulating in the culturing system and thereby affecting the growth of cells. New medium can then be introduced to the chamber via the inlet.

In one embodiment, gases such as air, oxygen and carbon dioxide are provided to the cells in the form of dissolved gases in the culture medium. In one embodiment, gases are infused to the culture medium by directly exposing the culture medium to the gases. For example, when culture medium flows into a tank wherein the headspace in the tank is filled with a mixture of air and 5% CO₂, gas exchange between the culture medium and headspace occurs. The resulting culture medium is then supplied to the cells in the present cell culture system.

In one embodiment, cell culture medium, reagents and gases introduced into the cell incubation chamber are all filtered in advance using appropriate filter (e.g. pore size of 220 nm) to remove bacteria or other undesirable microorganisms from entering the cell culture system and thereby contaminating the cells. In one embodiment, culture medium infused with atmospheric air with 5% CO₂ and 20% O₂ are filtered and then introduced into the cell incubation chamber. In one embodiment, cells are incubated at 37° C. In one embodiment, cells are incubated with continuous perfusion of 5% CO₂/20% O₂ provided by the culture medium. In one embodiment, culture medium is renewed every three days.

In one embodiment, volume of culture medium for cell culture depends on a number of factors such as the size of the cell incubation chamber, type of cells being cultured and type of assays to be carried out. In one embodiment, volume of culture medium is 100 ml.

In one embodiment, cell-containing microgels are dispersed in the cell incubation chamber such that the microgels are arranged in a specified manner. In one embodiment, the cell incubation chamber is configured with U-shape arrays to hold microgels in an ordered array (FIG. 3, right panel).

In one embodiment, the cell incubation chamber is configured with rows of anchoring structures for anchoring the droplets (or microgels) at pre-determined positions in the droplet incubation chamber. The anchoring structures may take the form of pillars such as posts arranged in a way that is capable of trapping individual microgels (FIG. 8), or grooves which trap individual microgels by interfacial tension (FIG. 9).

In one embodiment, cell-containing microgels are dispersed in the cell incubation chamber such that the microgels are randomly packed.

Overall, the present invention provides a novel approach for single-cell culture and analysis. The conventional approach for studying single cell using microfluidic techniques is to encapsulate each cell in a water-in-oil emulsion. However, since the small aqueous compartment containing cells are dispersed in the oil phase, new reagents or fresh culture medium cannot be supplemented to the small aqueous compartment in the presence of an outer layer of the oil phase, making continuous cell culture not feasible.

This invention is particular useful when digital analysis of the cellular content is required or an absolute quantity of a target molecule in a cell is of interest. When digital detection and quantitation of target molecule in each cell), existing digital platforms are limited to end-point detection (i.e., detection after end of reactions) and one single type of reaction and detection (e.g., digital PCR reactions and digital ELISA reactions cannot be integrated into one platform such that PCR reactions and ELISA reactions can be carried out in different droplets). For target molecules that have multiple copies in a cell such as RNA and proteins (as opposed to a gene or a single nucleotide polymorphism (SNP) which usually exists in the genome of the cell as a single copy), end-point detection is not able to quantify these target molecules with precision. These existing platforms may differentiate the types of target molecules (e.g. different species of mRNA) but cannot determine the exact copy number of each species of the target molecules. Therefore, these current digital platforms cannot detect multiple biomolecules in a real-time manner and cannot monitor phenotypic properties and genotypic properties of the cells simultaneously.

In this invention, by using hydrogels for encapsulating individual cells into microgels rather than using a water-in-oil emulsion, cell culture medium can be supplemented to the encapsulated cells and wastes can be removed from the cells through the pores on the microgels, thereby permitting each cell to survive and grow continuously in the incubation chamber. Likewise, reagents and washing buffers that are necessary for cell culture or assays can be supplemented to each cell, thereby permitting real-time monitoring of various phenotypic and/or genotypic characteristics at single-cell level. Since different types of reagents are required for detecting different types of biomolecules (such as genotypic biomarkers like RNA or DNA and phenotypic biomarkers like), multiple steps for adding reagents and washing are necessary in order to label and detect these different types of biomolecules on the same platform. Since the present microgels are permeable to aqueous solutions, provision of assay reagents or washing buffers to the encapsulated cells and their removal from the encapsulated cells can be much simplified. Further, as provided herein, the present invention is equipped with special optic system for detecting a wide range of signals from the cells, thereby allowing detection of signals representing different target molecules simultaneously. Taking the above-mentioned advantages together, the present invention permits a cell culture of cells at single-cell level and a real-time detection of multiple target molecules in each cell in a simpler and more efficient manner.

### Applications of the Present Single-Cell Culturing System

There are systems for partitioning a population of cells into droplets or microgels. These prior systems may be used to study phenotypic or genotypic characteristics of cells through end-point measurement of target biomolecules or taking end-point morphological images of the cells but are not capable of on-going cell culture and analysis of the cells.

In contrast, the present invention provides a single-cell culturing system which does not only prepare microgels containing single cells and detect target biomolecules present in each cell, but also permits continuing cell culture and continuing monitoring of events occur in the cells and studying phenotypic or genotypic characteristics of these cells at single-cell level in a real-time manner.

For example, to determine the quantity of messenger RNA (mRNA) or microRNA (miRNA) in a particular type of cell as it goes through various stages of cell cycle, development, or differentiation, existing systems require multiple steps for preparing cell samples obtained at different time points (i.e. stages of cell cycle/development/differentiation), purification of nucleic acids from the each cell sample to get multiple nucleic acid samples, and determine the quantity of mRNA or miRNA using rt-PCR reactions for each nucleic acid sample. In contrast, by culturing the cells in an on-going manner in an environment simulated to conventional culture systems, quantity of mRNA or miRNA at different stages of cell cycle, development, or differentiation can be monitored in a real-time manner as the cells grow or differentiate in the present culturing system. This approach is particularly useful for studying variations in the quantity of target biomolecules during the growth or differentiation of cells since it reduces cell-to-cell or batch-to-batch variations and hence improves the accuracy of quantification and analysis. The present culturing system also simplifies the procedures, saves time and labor, and reduces the risk of sample loss and contamination. Therefore, the present culturing system provides a more accurate and efficient means for studying cellular events and characteristics of cells.

By allowing cell-containing droplets to be incubated independently, it is possible to study the response or responsiveness to stimuli of each cell in a real-time and continuous manner as the cells grow in the incubation chamber. As illustrated in FIG. 3 and Example 4, agarose is used to create hydrogel matrix and encapsulate individual cell molecules in microgels. The microgels are then loaded into the present cell incubation chamber for incubation and real-time monitoring.

In one embodiment, the present invention is used to study response to drug or treatment of cells at single-cell level. For example, the present invention can monitor responsiveness to a chemotherapeutic agent (e.g. doxorubicin, paclitaxel) of each cancer cell within a microgel by adding the chemotherapeutic agent to the culture medium and measure signals (e.g. fluorescent probes indicative of cell viability) from each microgel at various time points. Example 5 describes one example of using the present invention for monitoring single cell response to anti-cancer drug doxorubicin hydrochloride (Dox).

In one embodiment, the present single-cell culturing system is used to culture individual molecules of cell, spheroid or organoid. Spheroids, consisting of an aggregation of cells, present a three-dimensional cell modeling that simulates a live cell's environment conditions. Spheroids conserve molecular signals and phenotypes, making them ideal for drug screening, especially in the personalized medicine development. On the other hand, organoids are collections of organ-specific cell types that are derived from one or a few types of cells (e.g. progenitor cells) and possess native tissue structures of a given organ, thereby representing a superior model of in vivo situation.

In one embodiment, the present single-cell culturing system is used to prepare and culture single-cell derived spheroids from patient-derived cells from human tissues or biofluids. The heterogeneity of microenvironment and responsiveness to chemotherapeutic stimuli of single-cell derived spheroids can be monitored and evaluated in a real-time manner. Example 6 describes one example of using the present invention to prepare spheroids from a single human breast cancer cell and culture the spheroids in a microgel setting. Example 7 describes one example of using the present invention to monitor microenvironments within spheroids. Example 8 describes one example of using the present invention to monitor response to drug of spheroids.

In one embodiment, the present single-cell culturing system is used to prepare and culture single-cell derived organoids from patient-derived cells from human tissues or biofluids. The heterogeneity of microenvironment and responsiveness to chemotherapeutic stimuli of single-cell derived organoids can be monitored and evaluated in a real-time manner. Examples 6-8 describing procedures for analyzing single cell-derided spheroids are also applicable for analyzing single cell-derided organoids.

### Exemplary Applications

This description provides a number of examples to illustrate uses of the present invention for detecting or monitoring cellular events or molecules at single-cell level for various purposes. The following are exemplary description illustrating how the present invention can be used to monitor a wide range of cellular events or examine a wide range of cell characteristics at single-cell level and in a real-time manner. However, one skilled in the art will readily appreciate that the examples and description provided are merely for illustrative purposes and are not meant to limit the scope of the invention which is defined by the claims following thereafter.

### Monitoring of Cellular and Biochemical Events Occur in a Cell

The present invention can be used to detect molecules that are indicative of events occurred within a single cell, or indicative of phenotypic and genotypic characteristics of cells at single-cell level and in a real-time manner, thereby allowing monitoring these events and characteristics in each cell continuously.

In one embodiment, the detection or monitoring is conducted in a qualitative manner.

In one embodiment, the detection or monitoring is conducted in a semi-quantitative, relative quantitative or absolute quantitative manner.

In one embodiment, the present invention further measures the quantity of these indicative molecules in each cell in a real-time manner. This will provide valuable quantitative information for subsequent in-depth analysis and is particularly useful for investigating dose-response relationship, or prognosis or diagnosis that is primarily based on reference values.

In one embodiment, the present invention is able to detect or monitor any event within a cell. In one embodiment, event is a cellular event or a biochemical event. In one embodiment, event is an event that occurs at any point during the initiation or progression of a physiological process such as cell cycle, cell differentiation and immune response. In one embodiment, event is an event that occurs at any point during the initiation or progression of a disease. In one embodiment, event is a response to environmental stimuli or stress such as endoplasmic reticulum (ER) stress, mechanical stress, hypoxia and oxidative stress. In one embodiment, event is a response to chemical stimuli including chemotherapeutic stimuli.

### Detection, Examination and Monitoring of Phenotypic and Genotypic Characteristics of Cells

In one embodiment, the present invention provides a method for detecting, examining and monitoring phenotypic and genotypic characteristics of cells at single-cell level and in a real-time manner.

In one embodiment, phenotypic characteristics are any observable traits of a cell. In one embodiment, phenotypic characteristics include but are not limited to responses to chemical or environmental stimuli, profile of secreted proteins and profile of biomarkers on cell membrane.

In one embodiment, genotypic characteristics include but are not limited to nucleotide sequence, alteration, insertion or deletion of nucleotide sequence, which can be coding or non-coding sequence, DNA or RNA. In one embodiment, genotypic characteristics are genomic size, copy number of a particular target, absolute or relative position of a target in the genome, or any information about a particular sequence unit in the genome.

In one embodiment, the present invention provides a method for detecting gene variation at single-cell level. Example 9 describes an on-chip detection of gene variation in each tumor cell on the cell incubation chamber.

In one embodiment, phenotypic characteristics and genotypic characteristics are detected, examined or monitored concurrently in the same droplet incubation chamber. In one embodiment, phenotypic characteristics and genotypic characteristics are detected, examined or monitored separately, which can be conducted in the same droplet incubation chamber one after the other.

In one embodiment, after examination of phenotypic characteristics of cells, the genomic heterogeneity of singe cell is interrogated with the same incubation chip. Example 11 provides one example showing the present method is capable of interrogating the phenotypic and genotypic characteristics of single cell in tandem, which surely advances the understanding of the molecular mechanisms underlying cellular function and dysfunction.

Detection and Quantification of Single-Cell mRNA and miRNA

In one embodiment, the present invention provides a method for detecting and quantifying total or specific messenger RNA (mRNA) at single-cell level and in a real-time manner.

As illustrated in FIG. 1 and Example 2, lysis buffer, RT-PCR mixer, primer, TaqMan™ probes or other reagents for RT-PCR are loaded into the droplets along with cells for mRNA detection and quantification of single cell via RT-PCR. The lysis buffer is delicately selected to minimize the inhibitive effect of RT-PCR, since no washing step will be performed before RT-PCR. In one embodiment, IGEPAL CA-630 and bovine serum albumin performed are chosen as lysis buffer as they are better than sodium dodecyl sulfate and other detergent-based lysis buffer. During the PCR process, real-time monitoring of the fluorescent signals of individual droplets is carried out to obtain time-series of fluorescence images by an optic system. The method described herein monitors the amplification of targeted DNA molecules during the PCR in a real-time rather than only at the end of the PCR process as in conventional PCR and digital PCR system. The fluorescence images are then analyzed by an image processing software to compute and assign to variations of fluorescent signal intensities of individual droplets as a function of time during the PCR process which enables detection and quantification of specific mRNA in single cell resolution.

In one embodiment, the present invention provides a method for detecting and quantifying total or specific microRNA (miRNA) at single-cell level and in a real-time manner. This method can reveal the types and quantification of microRNA (miRNA) at single cell level. Example 3 illustrates one example of using the present invention to detect and quantify miRNA in single exosomes.

In one embodiment where the cells are cultured using the present invention, quantity of mRNA and miRNA of cells can be determined in an on-going and real-time manner and variations in their quantities throughout the culturing process can be monitored.

### Real-Time Monitoring of Drug Response at Single-Cell Level

In one embodiment, the present invention provides a method for monitoring cell response to a chemical, a therapeutic agent or an external stimulus at single-cell level and in a real-time manner.

Therapeutic agents or drugs to be studied herein can be therapeutic molecules of any nature or type, regardless of the type and stage of diseases they intend to treat.

In one embodiment, drug response is measured based on parameters that are indicative of drug efficacy, level of physiological or biochemical activities in the cells, cell viability, level of biomolecules target or affected by the drug, level of drug molecule in its original form or metabolized form, level of drug metabolites or by-product and the like. A skilled person in the art would be able to select appropriate parameters for examining a drug response for a particular drug molecule.

Example 5 shows one example of using the present invention to study cell responses of tumor cells to an anti-cancer drug. Example 8 shows one example of using the present invention to study cell responses of single-cell derived spheroids to an anti-cancer drug.

Example 10 describes one example of using the present invention to study genetic information of single cell after the analysis of drug responses describe herein.

Monitoring Microenvironments within Single-Cell Derived Spheroids

In one embodiment, the present invention provides a method for monitoring the microenvironment within a single-cell derived spheroid in a real-time manner.

In one embodiment, microenvironment refers to chemical factors exist in a cell, a membrane-bound organelle such as exosome, a spheroid or an organoid, including but are not limited to pH, type and level of ions, type and level of reactive oxygen species, level of oxygen, level of carbon dioxide, level of nutrients (e.g. minerals, vitamin, amino acids) and the like.

Example 7 illustrates one example of using the present invention to monitor hypoxia condition in a spheroid.

In one embodiment, the present invention provides a microfluidic cell culturing system for monitoring a plurality of cells or cellular structures in a real-time manner, the system comprises
a) a cell incubation chamber for culturing cells, which comprises an array of anchoring structures, each anchoring structure for holding and independently culturing no more than one cell or one cellular structure encapsulated in a microgel particle which comprises pores for fluids to move into and out of the particle;
b) one or more inlets for introducing culture medium and other fluids into the cell incubation chamber any time during cell culturing;
c) one or more outlets for removing fluids from the cell incubation chamber any time during cell culturing;
d) a pumping unit for driving the flow of fluids within the system;
e) a temperature-controlling unit for regulating the temperature within the system;
f) a plurality of microfluidic channels for carrying fluids within the system; and
g) a detection unit for detecting signals from each cell or cellular structure in a real-time manner, the signals are associated with a cellular activity or characteristics of the cells or cellular structures.

In one embodiment of the present system, the characteristics are one or more of phenotypic characteristics, genotypic characteristics, and microenvironment conditions of the cells or cellular structures.

In one embodiment of the present system, the microenvironment conditions are pH, oxygen concentration, nutrient content, ionic concentration, electrical potential, or pressure. In one embodiment, the cellular activity is part of a signal transduction event.

In one embodiment of the present system, the cellular activity is cell cycle, cell differentiation, immune response, a response to an environmental stimulus, a response to stress or a response to a chemical stimulus. In one embodiment, the stress is endoplasmic reticulum stress, mechanical stress, hypoxia or oxidative stress.

In one embodiment of the present system, the signals indicate the presence of a target molecule which is associated with the cellular activity or characteristics. In one embodiment, the target molecule is nucleic acids, peptides, proteins, enzymes, small molecules or ions.

In one embodiment of the present system, the target molecule is labelled with signal-generating probes, thereby producing signals indicating the presence of the target molecule.

In one embodiment of the present system, reagents for labelling the target molecule are introduced to the cell incubation chamber via one or more inlets, the reagents enter the microgel particle and label the target molecule in the cells or cellular structures in the cell incubation chamber.

In one embodiment of the present system, detection of signals is performed continuously or intermittently when the target molecule is being labelled. In one embodiment, signals are detected and converted into digital values to obtain the total number of the target molecule in each of the cells or cellular structures.

In one embodiment, the detection unit comprises a charge-couple device.

In one embodiment of the present system, the cellular structures are spheroids or organoids.

In one embodiment of the present system, the microgel particle is composed of a hydrogel matrix.

In one embodiment of the present system, the microgel particle is produced by a droplet generating device which comprises a structure consisting of a flow focusing structure, a crossflowing structure, a co-flowing structure, a step emulsion structure or a microchannel emulsification structure.

In one embodiment of the present system, the microgel particle has a diameter in the range of 10 μm to 200 μm.

In one embodiment, the present invention provides a method for monitoring a cellular activity or characteristics of a plurality of cells in a real-time manner, the method comprises the step of culturing the cells and determining the absolute quantity of a molecule in said cells using the microfluidic system of this invention, and the molecule is associated with the cellular activity or characteristics.

In one embodiment, the present invention provides a method for culturing and counting target molecules in a plurality of cells or cellular structures in a real-time manner, the method comprises the steps of
a) providing to a microfluidic cell culturing system a plurality of cells or cellular structures encapsulated in microgel particles, each particle contains no more than one cell or cellular structure, the microfluidic cell culturing system comprises a cell incubation chamber, the chamber comprises an array of anchoring structures, each anchoring structure holding no more than one microgel particle;
b) culturing the cells or cellular structures in the cell incubation chamber with continuous perfusion of culture medium;
c) providing to the cell incubation chamber reagents for labelling target molecules of the cells or cellular structures;
d) allowing the reagents to label the target molecules, producing fluorescent signals;
e) detecting fluorescent signals from the microgel particle; and
f) converting the signals into digital values to obtain the total number of the target molecules in each cell or cellular structure.

In one embodiment of the present method, the plurality of cells exists in the form of a plurality of spheroids or a plurality of organoids. In one embodiment, each of the microgel particles contains no more than one cell, one spheroid or one organoid.

In one embodiment of the present method, the microgel particles are produced by providing to a droplet generating device a suspension of cells and a hydrogel solution, the droplet generating device comprises a structure consisting of a flow focusing structure, a crossflowing structure, a co-flowing structure, a step emulsion structure or a microchannel emulsification structure. In one embodiment, the droplet generating device is part of the microfluidic device.

In one embodiment of the present method, the microgel particle has a diameter in the range of 10 μm to 200 μm.

In one embodiment of the present method, where in step (c), reagents are provided to the cell incubation chamber via one or more inlets of the microfluidic device, the reagents enter the microgel particles and label the target molecules of cells in the cell incubation chamber.

In one embodiment of the present method, the detection of said signals is performed continuously or intermittently when the target molecule is being labelled.

In one embodiment of the present method, fluorescent signals are detected by an optic system.

In one embodiment of the present method, the method detects 1-10 types of target molecules. In one embodiment, the target molecule is nucleic acids, peptides, proteins, enzymes, small molecules or ions.

In one embodiment of the present method, the total number of target molecules in each cell is indicative of one or more cellular activities occurred in the cell or one or more characteristics of the cell. In one embodiment, the characteristics are one or more of phenotypic characteristics, genotypic characteristics, and microenvironment conditions of the cells or cellular structures. In one embodiment, the microenvironment conditions are pH, oxygen concentration, nutrient content, ionic concentration, electrical potential, or pressure. In one embodiment, the cellular activities are part of a signal transduction event.

In one embodiment, the cellular activities are cell cycle, cell differentiation, immune response, response to an environmental or chemical stimulus, or response to stress. In one embodiment, the stress is endoplasmic reticulum stress, mechanical stress, hypoxia or oxidative stress.

Throughout this application, various publications are cited. The disclosures of these publications in their entireties are hereby incorporated by reference into this application to more fully describe the state of the art to which this invention pertains.

Throughout this application, it is to be noted that the transitional term “comprising”, which is synonymous with “including”, “containing” or “characterized by”, is inclusive or open-ended, and does not exclude additional, un-recited elements or method steps.

This invention will be better understood by reference to the examples which follow. However, one skilled in the art will readily appreciate that the examples provided are merely for illustrative purposes and are not meant to limit the scope of the invention which is defined by the claims following thereafter.

### EXAMPLES

### Example 1—Construction of Droplet Generator and Droplet Incubation Chamber

The microchips of the droplet generating device and droplet incubation chamber are made of polydimethylsiloxane (PDMS), silicon, or plastics (e.g. polycarbonate, cyclic olefin copolymer (COC)). For the PDMS microchip fabrication, the structure is photolithographically patterned on the silica substrate using SU-8 photoresist (Microchem) to form the mold. The mold is used to fabricate PDMS replicas. The PDMS replicas are bonded with cover glass using plasma treatment to form the PDMS chip. The surface of chip is treated with fluorosilane (Aquapel) to gain the hydrophobicity. The silicon chip is fabricated in similar way. The silicon wafer with pattern etched was bonded with a glass wafer with inlets and outlets drilled using the anodic bonding technique. The bonded silicon wafer is diced into individual chips. The surface of silicon chip is treated with fluorosilane (Aquapel) to gain the hydrophobicity.

### Example 2—Real-Time Multiplexed Detection and Quantification of Single-Cell mRNA Expression

Cancer cells were treated with 0.25% Trypsin-EDTA (Thermofisher Scientific, USA) to prepare cells suspension dispersed in the PBS solution (pH 7.4). The cell concentration was determined by manual cell counting and diluted to the desired concentration. The final cell solution was supplemented with 17% OptiPre Density Gradient Medium (Sigma-Aldrich, USA), and 1% (v/v) Pluronic F-68 (Life Technologies). The lysis buffer consisted of 10 mM Tris (pH 7.4), 0.25% IGEPAL CA-630 (Sigma-Aldrich) and 0.1% bovine serum albumin (BSA, Sigma-Aldrich) which showed less suppression effect on the post RT-PCR than other detergent-based lysis buffers was used. The RT-PCR mix was composed of 1× Reaction Mix, 1× Enzyme Mix (Thermo Fisher, 12574030, SuperScript III One-Step™), primers, and one or up to four fluorescence-labelled TaqMan™ probes supplemented with droplet stabilizer. The oil phase can be mineral oil, silicon oil or fluorinated oil, mixed with 1%, 2.5% or 5% (w/w) surfactants. The cell suspension, lysis buffer, RT-PCR mix and oil phase was loaded into droplet generator to isolate individual cell in droplets for RT-PCR, as shown in the left panel of FIG. 1. The droplets containing individual cell were loaded into the droplet incubation chamber. The droplet incubation chamber was loaded on temperature-controlled plate, equipped with an optic system above the plate to capture the fluorescence signal of droplets during PCR process by taking the images at specific time intervals. The temperature of the plate was programed to complete the processes, as shown in right panel of FIG. 1. The temperature of the plate was set at 37° C. for 15 min to release mRNA from cell membrane encapsulation, subsequently at 50° C. for 30 min to synthesis cDNA of the mRNA via the reverse transcription, and 94° C. for 3 min (initial denaturation), 29 cycles of 94° C. for 30 s, 54° C. for 30 s, and 65° C. for 30 s, followed by a single final extension for 5 min at 65° C. during the PCR process. Apart from fluorescent at the end-point of PCR, the scanning of the incubation chips was also taken every three minutes via the optic system during the PCR process. The time-series fluorescence images of incubation chips were analyzed by the image processing software to assign to variations of fluorescent signal intensities of individual droplets during PCR process, as shown in FIG. 4. The types and relative quantities of specific mRNA in each cell were determined by the fluorescent signal intensities curve of corresponding droplets via time.

### Example 3—Real-Time Multiplexed Detection and Quantification of miRNA in Single Exosome

Exosomes extracted from biofluids like, blood or urine, were dispersed in in the PBS solution (pH 7.4) and diluted to desired concentration, supplemented with 1% (v/v) Pluronic F-68 (Life Technologies). The lysis buffer consisted of 10 mM Tris (pH 7.4), 0.25% IGEPAL CA-630 (Sigma-Aldrich) and 0.1% bovine serum albumin (BSA, Sigma-Aldrich). The RT-PCR mix was composed of 1× Reaction Mix, 1× Enzyme Mix (Thermo Fisher, 12574030, SuperScript III One-Step™), primers, and one or up to four fluorescence-labelled TaqMan™ probes supplemented with droplet stabilizer surfactants. The oil phase can be mineral oil, silicon oil or fluorinated oil, mixed with 1%, 2.5% or 5% (w/w) surfactants. The exosome suspension, lysis buffer, RT-PCR mix and oil phase was loaded into droplet generator to isolate individual cell in a single droplet for RT-PCR. The droplets containing individual exosome were loaded into the incubation chamber. The RT-PCR process, optic detection and image-processing were performed as the method in the Example 2. The types and relative quantities of specific miRNA in each exosome were determined by the fluorescent signal intensities curve of corresponding droplets via time.

In another embodiment, TaqMan™ Advanced miRNA cDNA Synthesis Kit (Thermo Fisher, USA) may be used for the reverse transcription to synthesize complementary DNA (cDNA) from messenger RNA (mRNA) and TaqMan™ Advanced miRNA Assay (Thermo Fisher, USA) is used for the detection of specific sequences in the real-time PCR.

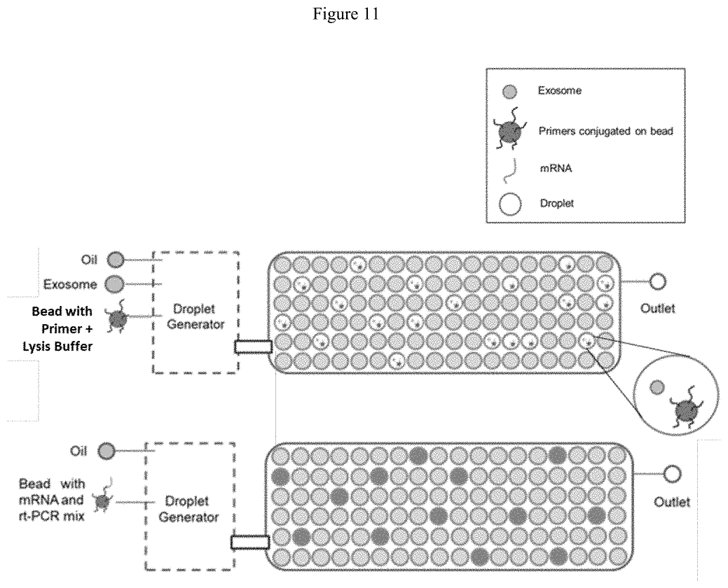

For absolute counting of the RNA from a single cell or exosome, a system such as shown in FIG. 11 can be used, involving two rounds of encapsulation: one for single exosome, and the other for single mRNA molecules.

As shown in the upper panel of FIG. 11, a single exosome, magnetic beads conjugated with primer specific to the target RNA and lysis buffer for lysing the exosome are encapsulated into one droplet by the droplet generating device. Droplet generation may take the form shown in FIG. 6. After droplets are generated, they are stored in the droplet storage chamber at the right. In each isolated droplet, the single exosome is lysed and the RNA contained therein is released and paired with the target-specific primer on the magnetic beads. Droplets are then collected from the outlet for subsequent analysis. All the collected droplets are then broken using a solvent (e.g. Perfluoro-1-octanol) to dissolve the oil phase and obtain an aqueous suspension of the magnetic beads with RNA from the single cell/exosome. A washing solution (e.g. PBS) is then added to the suspension followed by mixing with vortex. The mixture is then allowed to settle on a magnetic shelf. Components that are not necessary for subsequent rt-PCR reactions would be removed together with the washing solution by pipetting while the magnetic beads with primer conjugated with the target RNA are retained.

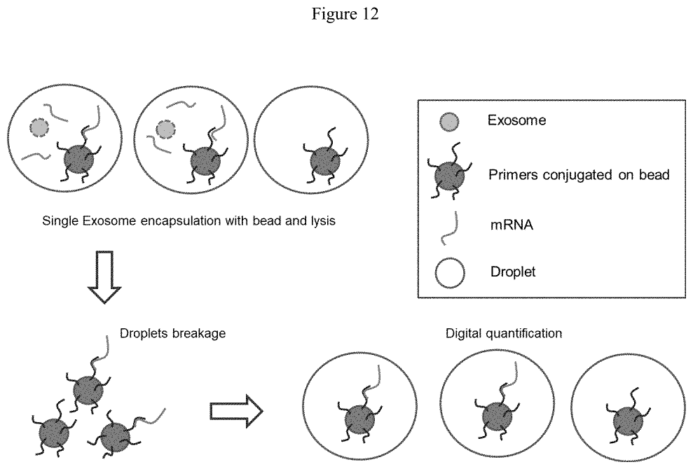

As shown in FIG. 12, mRNA molecules from one exosome are released after lysis of the exosome and will be conjugated with a target primer on the magnetic beads. Unnecessary components are washed away through washing steps. The resulting sample containing the beads with primer conjugated with mRNA will then be encapsulated into droplets for digital quantification of the mRNA.

As shown in the lower panel of FIG. 12, the magnetic beads with primer conjugated with mRNA are mixed with a reverse transcription mixture and PCR mixture (collectively rt-PCT mix) and then loaded into an integrated droplet microfluidic system for in situ reverse transcription and PCR thermal cycling. Droplet generation may take the form shown in FIG. 5 since reverse transcription and PCR are hot start reactions and therefore the mRNA sample and rt-PCT reaction mix can be pre-mixed before encapsulation. Fluorescent signals (as indicated by the darker dots in the droplet storage chamber in the lower penal of FIG. 11) are then detected digitally through a microscopic camera and absolute count of the RNA target from a single exosome can be calculated.

### Example 4—Compartmentalizing Individual Cell in Agarose Microgels for Long-Term Incubation

Cancer cells were treated with 0.25% Trypsin-EDTA (Thermofisher Scientific, USA) to prepare cells suspension dispersed in the PBS solution (pH 7.4). The cell concentration was determined by manual cell counting and diluted to a desired concentration. The final cell solution was prepared by supplemented with 17% OptiPre Density Gradient Medium (Sigma-Aldrich, USA), 0.1 mg/ml BSA (Thermofisher Scientific, USA) and 1% (wt/v) Pluronic F-68 (Life Technologies).

A 3% (w/v) low-melting point agarose solution (Sigma-Aldrich, USA) was heated to 60° C. for 10 minutes before use to completely dissolve the agarose, and the syringe and connecting lines containing the agarose solution during the injection were wrapped by a wire sleeve to maintain the agarose solution at 60° C.

Then, the prepared cells suspension, the agarose solution of 3% (w/v) and fluorinated oil HFE7500 (3M, USA) containing 2% PFPE-PEG-PFPE were injected to a droplet generator by a syringe pump to obtain gel droplets. As shown in FIG. 3, the cell suspension was introduced from the middle cannel, the agarose solution was introduced from the top channel the oil phase, such as 2% PFPE-PEG-PFPE fluorinated oil HFE7500, was introduced from side channels to form gel droplets. The agarose solution and cell solution were packaged in a droplet and suspended in the oil phase of HFE 7500, as shown in FIG. 3.

The hot agarose solution was mixed with the cell solution upon droplet generation. The temperature of the mixture was rapidly cooled to gelatinize and form a solid microgel particle. In order to avoid excessively high temperature in the droplet that affects the cell growth, the flow ratio of the cell solution to the agarose solution should be maintained at above 2:1, such that the internal temperature would not affect the normal growth of cells when the droplets were formed.

Loading and releasing of microgels and cell cultures were performed on the cell incubation chamber made of a microfluidic chip, as shown in the right panel of FIG. 3. The microgels were loaded into a cell incubation chamber with U-shape arrays to trap the microgel particles. The oil phase was removed by the air and the residual surfactants PFPE-PEG-PFPE and HFE7500 were then rinsed off by introducing a low boiling point fluorinated oil HFE7100 (3M, USA) into the cell culture chamber, then the HFE7100 was blown away from the cell incubation chamber by air. Due to the low boiling point (61° C.) of HFE7100, it was easier for air to remove HFE7100 thoroughly. Finally, the cell incubation chamber was filled with a cell culture medium (a liquid culture medium containing the substance required for the cells, the culture medium was any suitable culture medium, containing any suitable nutrient or detection reagent for subsequent testing), to rinse microgel particles on the chip at a high flow rate of 2 ml/min to remove any residual oil phase (just in case) in the cell incubation chamber, then the flow rate of culture medium was adjusted to the normal level of 200 μl/min, driven by a peristaltic pump. The whole culture system was kept at 37° C. under 5% CO₂ atmosphere.

Those skilled staffs in the art would imagine that any suitable gel material can be used to package a single cell for different cell cultures, for example, for commonly used agarose of different melting points, although the melting points are different, as long as adjusting the flow rate, flow ratio of cell solution and gel solution, the temperature of the encapsulated cell droplets would not be raised to make the cells to die during encapsulation. In some embodiments, the gel generally becomes liquid when heated, and cools to a solid state. The solid outer shell also has tiny pores, such as capillary pores or micropore, through which the internal liquid can be exchanged with outside, for example, the exchange of nutrients, oxygen, carbon dioxide, and some test reagents, or the exchange of waste gas and waste liquid, so that cells can grow continuously and maintain their inherent activity. By this way, tests could be performed at any time, for example, cell-specific tests, internal reaction tests. There were many ways to let the gel become liquid by heating, like the entire droplet generator was kept at a relatively high temperature. At this temperature, cells would not die, but gels could be maintained at a liquid state. When the gel was in contact with a solution containing cells, that is, when encapsulated, the temperature could be lowered, to form single cell encapsulation. After the first encapsulation, the oil phase was packaged to form a droplet with double layers. When each droplet was dispersed, for example, after loading on anchor structures of a microfluidic chip, the oil phase was removed, such that only the microgels with cell encapsulated were retained to achieve continuous culture. This has a significant different effect from the direct package by oil phase. It is generally difficult to culture the cells by the oil phase package, especially continuous culture to keep cells alive for a long time. Because the packaged cells were not allowed to have exchanges of nutrients, waste liquid, waste gas after the oil phase package, so that the cells can only be stored in a short period of time and cannot achieve a verity of different tests. Some tests take a long time for a number of times with viable cell. However, the present invention is carried out in such a manner that long-term culture of single cells is possible, to achieve testing of many cells, such as testing of drugs, and testing of cell activity, etc.

### Example 5—Real-Time Monitoring of Single Cell Responses to Drug

Individual tumor cells (e.g. human breast cancer cell line (MCF-7)) were isolated in the microgels as described in Example 4. Anti-cancer drug doxorubicin hydrochloride (Dox) was selected. NucBlue® Live ReadyProbes® Reagent (Life technologies) was used to stain the cell nuclei to identify the cells. LIVE/DEAD® Cell Imaging Kit (Life technologies) was used to quantify cell viability. The culture medium containing the Dox at different concentrations and fluorescent probes were introduced to the incubator chip. Brightfield and fluorescence images of cells in microgels were captured at 1, 3, 6 and 9 hrs. The uptake of Dox was quantified by the fluorescent intensity of Dox analyzed from the time-lapse images of each cell. Apoptosis behaviors of each cell at the different Dox concentrations were evaluated by the probes of LIVE/DEAD® Cell Imaging Kit (Life technologies).

### Example 6—Single-Cell Derived Spheroids Formation on Chip

The spheroids conserve the molecular signals, and phenotypes, making them ideal for drug screening, especially in the personalized medicine development. Human breast cancer cells (MCF-7) were compartmentalized in the microgels as described in Example 4. The microgels size was ˜200 μm in diameter. Then, microgels containing one individual cell were loaded into the cell incubation chamber which may take the form of a chip and released into the culture medium. The cells were incubated in the fresh medium for 10-2.0 days, driven by a peristaltic pump. The whole culture system was kept at 37° C. under 5% CO₂. The growth of cells was examined every day under a phase-contrast microscope. The diameter of spheroid reached to ˜50 μm after about 10 days.

### Example 7—Monitoring Microenvironments within Single-Cell Derived Spheroids

The microenvironments within spheroids (like hypoxia, pH) were examined using fluorescent probes. In one embodiment, the single-cell derived spheroids were prepared and cultured as described in Example 6. When the spheroid size reached ˜50 μm-100 μm, culture medium containing 10 μM image-iT™ Hypoxia. Probe (Thermofisher Scientific, USA) was introduced into the cell incubation chamber with staining for 1 hr. Then, the fluorescence images of single-cell derived spheroids was taken on a Zeiss 710 confocal microscope. The hypoxic condition of each single-cell derived spheroids were indicated by the fluorescent intensity of images of spheroids.

### Example 8—Monitoring Single-Cell Derived Spheroids Response to Drug

Single-cell derived spheroids were cultured according to the method demonstrated in Example 6, When the spheroid size reached ˜50 μm-˜100 μm, anti-cancer drugs doxorubicin hydrochloride (Dox) was selected. NucBlue® Live ReadyProbes® Reagent (Life technologies) was used to stain the cell nuclei to identify the cells. LIVE/DEAD® Cell Imaging Kit (Life technologies) was used to quantify cell viability. The culture medium containing the Dox at different concentrations and fluorescent probes were introduced to the incubator chip. Brightfield and fluorescence images of cells in microgels were captured at 1, 3, 9, 12 and 24 hrs. The uptake of Dox was quantified by the fluorescent intensity of Dox analyzed from the time-lapse images of each cell. The size of spheroids was quantified by the fluorescence images of NucBlue® probes stained nucleus. Apoptosis behaviors of each single-cell derived spheroids were evaluated by the probes of LIVE/DEAD® Cell Imaging Kit (Life technologies).

### Example 9—Real-Time Detection of Single-Cell Genomic Gene Variation

Individual tumor cells (e.g. human breast cancer cell line (MCF-7)) were isolated in the microgels as described in Example 4. The incubation chamber was loaded on a temperature-controlled plate, equipped with an optic system above the plate to capture the fluorescent signals of the each microgels at the end-time of PCR. The cell lysis buffer (0.5% (w/v) lithium dodecyl sulfate, 10 mM EDTA and 4U of Proteinase K in TE buffer) was introduced to the cell incubation chamber which may take the form of a chip. The incubation chamber was heated at 50° C. for 30 min to release the genomic DNA and digest the lysates. Then, the microgel was washed with 2% (w/v) Tween 20 in water for one time, 100% ethanol for one time, and 0.02% (w/v) Tween 20 for five times. For amplification and detection, 500 μL of PCR solution containing 1× Invitrogen Platinum Multiplex PCR Master Mix (Thermo Fisher Scientific, USA), 400 nM primers and 200 nM TaqMan probes were introduced to the incubation chamber and the microgels were soaked in the solution for 30 min to be saturated with the PCR solution. The oil containing surfactants was injected into the incubation chamber to isolate the microgels by the oil phase. To perform the PCR, the temperature of the plate was set at 94° C. for 3 min (initial denaturation), 29 cycles of 94° C. for 30 s, 54° C. for 30 s, and 65° C. for 30 s, followed by a single final extension for 5 min at 65° C. At the end-time of PCR, the fluorescent images of the incubation chip were acquired by the optic system. The specific genomic DNA in each cell was determined by the fluorescent signals of each microgel.

### Example 10—Post-Analysis of Single-Cell Genetic Information after Drug Response Test

After drug response test in the Example 3 or Example 6, the cell lysis, PCR and detection of genomic DNA information in each cell were conducted as the method in Example 9. The genetic information and drug response of each cells could be correlated by their location in the incubation chip.

### Example 11—Post-Analysis of Single-Cell Genetic Information after Examination of the Phenotypic Characteristics

After examination of phenotypic characteristics of cells, the genomic heterogeneity of singe cell is examined within the same cell incubation chamber. The lysis buffer with Proteinase K is utilized to break the cell membrane and digest the lysates to release the genomic DNA. The released genomic DNA is still trapped within microgels while lysis buffers and digested cellular residues are washed away to reduce the chance of inhibition of PCR. PCR mix, primer and TaqMan™ probes are loaded into the microgels for PCR and detection. Fluorinated oil with surfactant are required to isolate the microgels in oil phase to prevent interferences between different microgels since microgels are melted in liquid phase during the thermal cycles of PCR. The fluorescent signals indicating the amplification of targeted DNA molecules are monitored in real-time to reveal the genomic DNA variations across the cell population. The genetic information and phenotypic characteristics such as drug responses of each cells could be correlated by their location in the cell incubation chamber.

### REFERENCES

1. P. Zhu and L. Wang, “Passive and active droplet generation with microfluidics: a review” Lab Chip, 2017, 17, 34-75.
2. Baret, Jean-Christophe. “Surfactants in droplet-based microfluidics.” Lab on a Chip 12.3 (2012): 422-433.
3. Liu, Chunchen, Xiaonan Xu, Bo Li, Bo Situ, Weilun Pan, Yu Hu, Taixue An, Shuhuai Yao, and Lei Zheng. “Single-exosome-counting immunoassays for cancer diagnostics.” Nano letters (2018).
4. Abbyad, Paul, et al. “Rails and anchors: guiding and trapping droplet microreactors in two dimensions.” Lab on a Chip 11.5 (2011): 813-821.
5. Huebner, Ansgar, et al. “Static microdroplet arrays: a microfluidic device for droplet trapping, incubation and release for enzymatic and cell-based assays.” Lab on a Chip 9.5 (2009): 692-698.
6. Nivedita Majumdar, Thomas Wessel, Jeffrey Marks, ‘Digital PCR Modeling for Maximal Sensitivity, Dynamic Range and Measurement Precision’(2015), PLoS ONE 10(3): e0118833.

1. A microfluidic cell culturing system for monitoring a plurality of cells or cellular structures in a real-time manner, comprising
a) a cell incubation chamber for culturing cells, comprising an array of anchoring structures, each anchoring structure for holding and independently culturing no more than one cell or one cellular structure encapsulated in a microgel particle which comprises pores for fluids to move into and out of said particle;
b) one or more inlets for introducing culture medium and other fluids into the cell incubation chamber any time during cell culturing;
c) one or more outlets for removing fluids from the cell incubation chamber any time during cell culturing;
d) a pumping unit for driving the flow of fluids within the system;
e) a temperature-controlling unit for regulating the temperature within the system;
f) a plurality of microfluidic channels for carrying fluids within the system; and
g) a detection unit for detecting signals from each cell or cellular structure in a real-time manner, wherein said signals are associated with a cellular activity or characteristics of said cells or cellular structures.

2. The system of claim 1, wherein said characteristics are one or more of phenotypic characteristics, genotypic characteristics, and microenvironment conditions of the cells or cellular structures.

3. The system of claim 2, wherein said microenvironment conditions are selected from the group consisting of pH, oxygen concentration, nutrient content, ionic concentration, electrical potential, and pressure.

4. The system of claim 1, wherein said cellular activity is part of a signal transduction event.

5. The system of claim 1, wherein said cellular activity is selected from the group consisting of cell cycle, cell differentiation, immune response, a response to an environmental stimulus, a response to stress and a response to a chemical stimulus.

6. The system of claim 5, wherein said stress is selected from the group consisting of endoplasmic reticulum stress, mechanical stress, hypoxia and oxidative stress.

7. The system of claim 6, wherein said signals indicate the presence of a target molecule which is associated with said cellular activity or characteristics.

8. The system of claim 7, wherein said target molecule is selected from the group consisting of nucleic acids, peptides, proteins, enzymes, small molecules and ions.

9. The system of claim 8, wherein said target molecule is labeled with signal-generating probes, thereby producing signals indicating the presence of said target molecule.

10. The system of claim 9, wherein reagents for labelling said target molecule are introduced to said cell incubation chamber via said one or more inlets, wherein said reagents enter said microgel particle and label said target molecule in said cells or cellular structures in the cell incubation chamber.

11. The system of claim 10, wherein detection of said signals is performed continuously or intermittently when the target molecule is being labeled.

12. The system of claim 11, wherein said signals are detected and converted into digital values to obtain the total number of said target molecule in each of the cells or cellular structures.

13. The system of claim 12, wherein the detection unit comprises a charge-couple device.

14. The system of claim 13, wherein said cellular structures are spheroids or organoids.

15. The system of claim 14, wherein said microgel particle is composed of a hydrogel matrix.

16. The system of claim 15, wherein said microgel particle is produced by a droplet generating device comprising a structure selected from the group consisting of a flow focusing structure, a cross flowing structure, a co-flowing structure, a step emulsion structure and a micro-channel emulsification structure.

17. The system of claim 16, wherein said microgel particle has a diameter in the range of 10 μm to 200 μm.

18-47. (canceled)

48. The system of claim 1, wherein the system further includes an outlet for exporting oil phase-packaged microgel particles.

49. The system of claim 48, wherein the outlet is in fluidic communication with a storage system, and the oil phase-packaged microgel particles that are exported from the outlet flow into the storage system for storage or culture.

50. The system of claim 1, wherein the system further comprising a heating unit, and the heating unit allows the gel to maintain a liquid state.
